(19) United States
(12) Patent Application Publication
(10) Pub. No.: US 2022/0388951 A1
(43) Pub. Date: Dec. 8, 2022

(54) METHOD FOR EVALUATING BROMINATED FLAME RETARDERS

(71) Applicant: ARKEMA FRANCE, Colombes (FR)

(72) Inventor: Jean-Luc DUBOIS, COLOMBES CEDEX (FR)

(21) Appl. No.: 17/771,598

(22) PCT Filed: Oct. 26, 2020
(86) PCT No.: PCT/FR2020/051932

§ 371 Date: Apr. 25, 2022

(30) Foreign Application Priority Data
Oct. 30, 2019 (FR) FR1912167

(51) Int. Cl. C07C 321/26; C07C 321/22; C07C 321/06

(52) U.S. Cl. CPC C07C 321/26; C07C 321/22; C07C 321/06

(57) ABSTRACT

A process for upgrading brominated flame retardants comprises a) provision of a plastic comprising a flame retardant A consisting of a carbon-based chain substituted by at least two bromine atoms; b) recovery of a composition comprising said flame retardant A; c) bringing said composition comprising said flame retardant A into contact, in an organic solvent, with a compound A′ selected from the group consisting of Na2S2, K2S2, a potassium or sodium alkyl xanthate, and a compound of formula Y—SH in which Y is an alkali metal, an alkaline earth metal, a metal selected from rare earth metals, a —C(NH2)═NH unit or a —C(NR1R2)=NR3 unit; in order to form a mixture C comprising a compound B consisting of a carbon-based chain substituted by at least two —SH functional groups; and d) purification of the mixture C obtained and recovery of said compound B.

### TECHNICAL FIELD

The present invention relates to a process for the upgrading of brominated flame retardants. More particularly, the present invention relates to a process for the upgrading of brominated flame retardants contained in a plastic. The present invention also relates to polythiol compounds which are obtained by virtue of the present process and can be used in various applications.

### TECHNOLOGICAL BACKGROUND OF THE INVENTION

Tens of tonnes of electrical appliances and insulating foams accumulate each year worldwide. The majority of these appliances contain plastics, the recycling of which is necessary to meet regulatory and environmental directives.

Processes for the recycling of plastic consisting essentially of polystyrene are known in particular. For example, the applications US 2017/0298207 and WO 2015/000681 describe processes for the recycling of polystyrene by the liquid route, that is to say involving the use of a solvent. Processes for the recycling of polyurethane, another plastic frequently used for the implementation of numerous devices, are also known. Mention may be made, for example, of Yang et al., Procedia Environmental Sciences, 16 (2012), 167-175, who describes the various methods for the recycling of polyurethane (chemical, physical or landfilling).

However, none of these recycling techniques takes into account all the constituents of the plastic. This is because the majority of plastics contain additives, more particularly flame retardants. These compounds are generally polybrominated hydrocarbons which decompose with difficulty. Their accumulation in the environment is problematic due to their toxicity related to the presence of the bromine atoms. The documents CN 109268849 and CN105713640 describe processes for the heat treatment of residues comprising brominated flame retardants. These processes possess the disadvantage of requiring a sizeable energy contribution to achieve the targeted temperatures (>500° C.). In addition, none of the processes makes it possible to recover the flame retardant and/or to upgrade it.

There thus exists a need for a process which makes it possible to recover and to upgrade the flame retardants contained in plastics while minimizing the environmental impact.

### SUMMARY OF THE INVENTION

According to a first aspect, the present invention relates to a process for the upgrading of brominated flame retardants comprising the stages of:
a) provision of a plastic comprising a flame retardant A consisting of a carbon-based chain substituted by at least two bromine atoms;
b) recovery of a composition comprising said flame retardant A;
c) bringing said composition comprising said flame retardant A into contact, in an organic solvent, with a compound A′ selected from the group consisting of Na₂S₂, K₂S₂, a potassium or sodium alkyl xanthate, and a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl, provided that R¹, R² and R³ are not simultaneously H; in order to form a mixture C comprising a compound B consisting of a carbon-based chain substituted by at least two —SH functional groups;
d) purification of the mixture C obtained and recovery of said compound B.

The present process makes it possible to recover the brominated flame retardants contained in a plastic and to upgrade them in various applications. The present process prevents the dispersing of the brominated compounds in the environment while upgrading all of the products resulting from the various stages of the process. The present invention thus provides an environmentally responsible process for upgrading plastic.

According to a preferred embodiment, the compound A′ is a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl, provided that R¹, R² and R³ are not simultaneously H; preferably, Y is an alkali metal. Advantageously, in the —C(NR¹R²)═NR³ unit, R¹ is H and R² and R³ are, independently of each other, C₁-C₁₀ alkyl. Preferably, in the —C(NR¹R²)═NR³ unit, R¹ is H and R² and R³ are, independently of each other, C₁-C₄ alkyl. In particular, in the —C(NR¹R²)═NR³ unit, R¹ is H and R²═R³═C₄ alkyl.

According to a preferred embodiment, the plastic consists of a thermosetting polymer selected from the group consisting of polyester, polyurethane, polyurea/polyurethane, phenol-formaldehyde resin, melamine resin, epoxy resin and polyimide; or the plastic consists of a thermoplastic polymer selected from the group consisting of poly(methyl methacrylate), acrylonitrile/butadiene/styrene (ABS), polyamide, polylactic acid, polyhydroxyalkanoate, polybenzimidazole, polycarbonate, polyethersulfone, polyoxymethylene, polyetheretherketone, polyetherimide, polyethylene, polyphenylene oxide, polyphenylene sulfide, polypropylene, polystyrene, poly(vinyl chloride), polyvinylidene fluoride (PVDF) and polytetrafluoroethylene (PTFE).

According to a preferred embodiment, stage b) comprises a stage of depolymerization or of dissolution of said plastic in order to recover a composition comprising said flame retardant A.

According to a preferred embodiment, stage c) is carried out at a temperature of from 20° C. to 150° C. and at a pressure of from 1 bar absolute to 15 bars absolute.

According to a preferred embodiment, the compound A′ is NaSH or KSH.

According to a preferred embodiment, stage c) is carried out in the presence of H₂S; preferably, the molar ratio H₂S/A′ is less than 0.4. The presence of H₂S makes it possible to promote the reaction between A′ and the brominated functional group carried by said flame retardant A.

According to a preferred embodiment, stage c) is carried out in a reactor and, prior to bringing said flame retardant A into contact with said compound A′, a stream comprising an inert gas is introduced into the reactor. The introduction of an inert gas into the reactor prior to bringing the flame retardant into contact with the compound A′ makes it possible to limit or to prevent the presence of oxygen in the reactor during stage c). It has been found that the presence of oxygen promoted the formation of impurities, such as disulfide compounds or sulfur-based polymers.

According to a preferred embodiment, said carbon-based chain of said flame retardant A comprises from 3 to 20 carbon atoms, preferably from 5 to 15 carbon atoms.

According to a preferred embodiment, said flame retardant A is a C₃-C₂₀ alkyl at least substituted by two bromine atoms, C₃-C₂₀ alkenyl at least substituted by two bromine atoms, C₃-C₂₀ alkynyl at least substituted by two bromine atoms, C₃-C₂₀ cycloalkyl at least substituted by two bromine atoms, C₃-C₂₀ cycloalkenyl at least substituted by two bromine atoms, C₆-C₂₀ aryl at least substituted by two bromine atoms or C₃-C₂₀ ether at least substituted by two bromine atoms.

According to a preferred embodiment, said flame retardant A is selected from the compounds A-1, A-2, A-3, A-4, A-5, A-6, A-7, A-8 and A-9

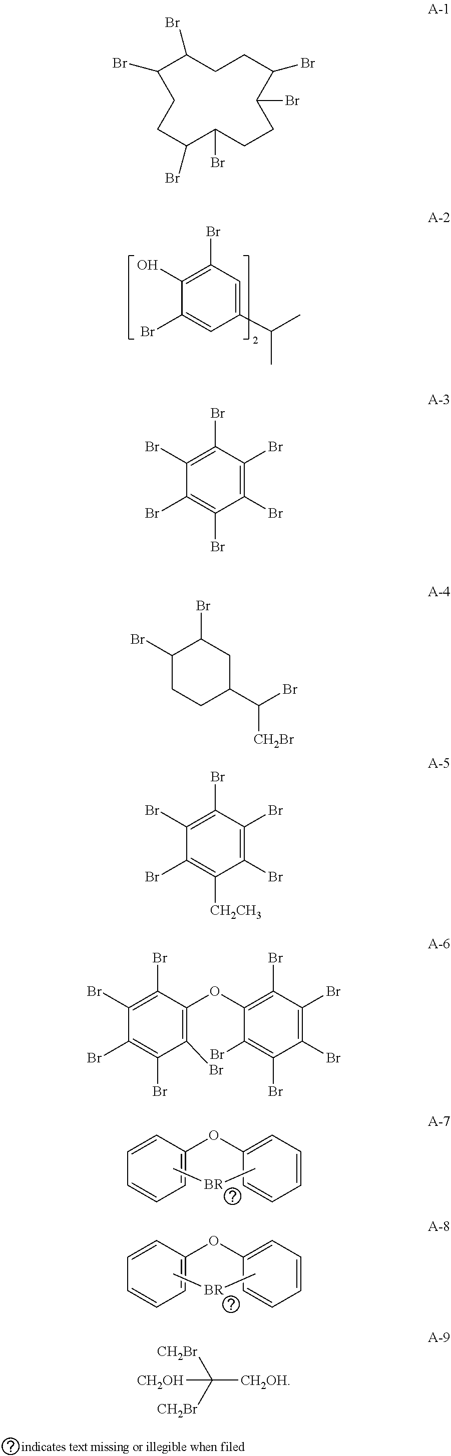

According to a preferred embodiment, said compound B is selected from the group consisting of the compounds B-1, B-2, B-3, B-4, B-5, B-6, B-7, B-8 and B-9

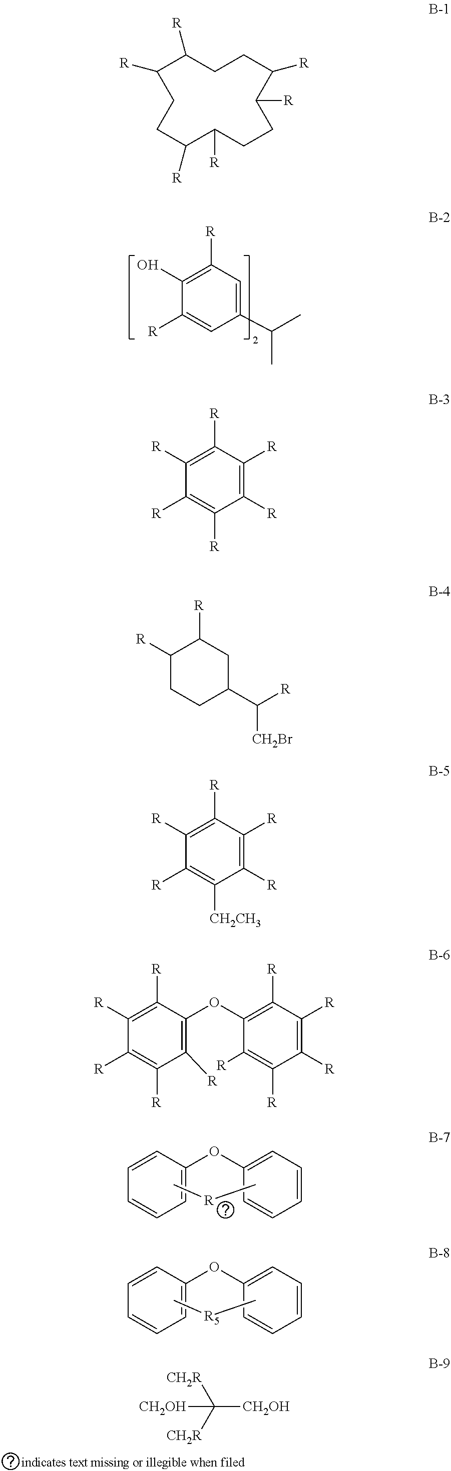

in which at least two R substituents are —SH; the other R substituents being, independently of one another, —SH, —Br, —OH, —H, ═S or forming a C═C double bond, by a reaction for elimination of H—R with R═OH or Br, between the carbon atom carrying the R substituent and a carbon atom adjacent to the latter carrying a hydrogen atom;
preferably, the other R substituents being, independently of one another, —SH, —Br, —OH or forming a C═C double bond, by a reaction for elimination of H—R with R═OH or Br, between the carbon atom carrying the R substituent and a carbon atom adjacent to the latter carrying a hydrogen atom;
more preferentially, the other R substituents being, independently of one another, —SH, —OH or forming a C═C double bond, by a reaction for elimination of H—R with R═OH or Br, between the carbon atom carrying the R substituent and a carbon atom adjacent to the latter carrying a hydrogen atom;
in particular, the other R substituents being, independently of one another, —SH or —OH.

The nature of the R substituent depends on the nature of the compound B. Thus, when the substituents are carried by an aryl group, the R substituent cannot be a double bond, H or ═S. In this case, the R substituents are selected from —SH, —Br or —OH.

According to a preferred embodiment, the compound A is the compound A-1 and the compound B is the compound B-1 in which at least two R substituents are —SH; preferably, the compound B is the compound B-1 in which at least three R substituents are —SH; more preferentially, the compound B is the compound B-1 in which at least four R substituents are —SH; in particular, the compound B is the compound B-1 in which at least five R substituents are —SH; more particularly, the compound B is the compound B-1 in which all the R substituents are —SH.

According to a second aspect, the present invention relates to a process for the preparation of a compound B comprising at least two —SH functional groups, characterized in that it comprises bringing a compound A consisting of a carbon-based chain substituted by at least two bromine atoms into contact, in an organic solvent, with a compound A′ selected from the group consisting of Na₂S₂, K₂S₂, a potassium or sodium alkyl xanthate, and a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl, provided that R¹, R² and R³ are not simultaneously H; said compound A being selected from the compounds A-1, A-2, A-3, A-4, A-5, A-6, A-7, A-8 and A-9 as defined above in order to form a compound B selected from the group consisting of the compounds B-1, B-2, B-3, B-4, B-5, B-6, B-7, B-8 and B-9 as defined above in which at least two R substituents are —SH.

According to a third aspect, the present invention relates to a compound B selected from the group consisting of the compounds B-1, B-2, B-3, B-4, B-5, B-6, B-7, B-8 and B-9 as defined above in which each R substituent is independently selected from —SH, —Br, —OH, —H, ═S or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;

preferably, in which each R substituent is independently selected from —SH, —Br, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;
more preferentially, in which each R substituent is independently selected from —SH, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;
in particular, in which each R substituent is independently selected from —SH and —OH; provided that at least two R substituents are —SH.

According to a preferred embodiment, said compound B is selected from the group consisting of the compounds B-1 and B-3 in which each R substituent is independently selected from —SH, —Br, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;

more preferentially, in which each R substituent is independently selected from —SH, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;
in particular, in which each R substituent is independently selected from —SH and —OH; provided that at least two R substituents are —SH.

According to a preferred embodiment, said compound B is selected from the group consisting of the compounds B-1, B-2, B-3, B-4, B-5, B-6, B-7 B-8 and B-9 as defined above in which the R substituent is —SH.

According to a fourth aspect, the present invention relates to the use of the compound B according to the present invention as crosslinking or vulcanizing agent, reactant for the preparation of sulfur-based compounds, such as thiourethanes, polysulfides and the like, chain-transfer agent, metal-complexing agent, ore flotation agent, antioxidant or heat stabilizer.

### DETAILED DESCRIPTION OF THE INVENTION

### Process for the Upgrading of Flame Retardants

According to a first aspect, the present invention relates to a process for the upgrading of brominated flame retardants. By virtue of the present invention, brominated flame retardants are recovered and converted into polythiols, it being possible for the latter to be used in numerous applications.

### Stage a) of the Process

The present process comprises the provision of a plastic comprising a flame retardant A consisting of a carbon-based chain substituted by at least two bromine atom.

Preferably, the plastic consists of a thermosetting polymer or of a thermoplastic polymer.

In particular, the plastic consists of a thermosetting polymer selected from the group consisting of polyester, polyurethane, polyurea/polyurethane, phenol-formaldehyde resin, melamine resin, epoxy resin and polyimide. Favorably, the plastic consists of a thermosetting polymer selected from the group consisting of polyester and polyurethane.

Alternatively, the plastic consists of a thermoplastic polymer selected from the group consisting of poly(methyl methacrylate), acrylonitrile/butadiene/styrene (ABS), polyamide, polylactic acid, polyhydroxyalkanoate, polybenzimidazole, polycarbonate, polyethersulfone, polyoxymethylene, polyetheretherketone, polyetherimide, polyethylene, polyphenylene oxide, polyphenylene sulfide, polypropylene, polystyrene, poly(vinyl chloride), polyvinylidene fluoride (PVDF) and polytetrafluoroethylene (PTFE). Favorably, the plastic consists of a thermoplastic polymer selected from the group consisting of poly(methyl methacrylate), acrylonitrile/butadiene/styrene (ABS), polyamide, polycarbonate, polyetheretherketone, polyethylene, polypropylene, polystyrene, poly(vinyl chloride), polyvinylidene fluoride (PVDF) and polytetrafluoroethylene (PTFE).

As mentioned above, said flame retardant A consists of a carbon-based chain substituted by at least two bromine atom. Said carbon-based chain of said flame retardant A advantageously comprises from 3 to 20 carbon atoms, preferably from 5 to 15 carbon atoms.

Preferably, said flame retardant A is a C₃-C₂₀ alkyl at least substituted by two bromine atoms, C₃-C₂₀ alkenyl at least substituted by two bromine atoms, C₃-C₂₀ alkynyl at least substituted by two bromine atoms, C₃-C₂₀ cycloalkyl at least substituted by two bromine atoms, C₃-C₂₀ cycloalkenyl at least substituted by two bromine atoms, C₆-C₂₀ aryl at least substituted by two bromine atoms or C₃-C₂₀ ether at least substituted by two bromine atoms. In particular, said flame retardant A is a C₅-C₁₅ alkyl at least substituted by two bromine atoms, C₅-C₁₅ alkenyl at least substituted by two bromine atoms, C₅-C₁₅ alkynyl at least substituted by two bromine atoms, C₅-C₁₅ cycloalkyl at least substituted by two bromine atoms, C₅-C₁₅ cycloalkenyl at least substituted by two bromine atoms, C₆-C₁₅ aryl at least substituted by two bromine atoms or C₅-C₁₅ ether at least substituted by two bromine atoms.

The term “alkyl” denotes a monovalent radical resulting from a linear or branched alkane comprising the number of carbon atoms specified. The term “cycloalkyl” denotes a monovalent radical resulting from a cycloalkane comprising the number of carbon atoms specified. The term “aryl” denotes a monovalent radical resulting from an arene comprising the number of carbon atoms specified. The term “alkenyl” denotes a monovalent radical comprising the number of carbon atoms specified and at least one carbon-carbon double bond. The term “alkynyl” denotes a monovalent radical comprising the number of carbon atoms specified and at least one carbon-carbon triple bond. The term “cycloalkenyl” refers to a monovalent radical resulting from a cycloalkene comprising the number of carbon atoms specified and at least one carbon-carbon double bond in its cyclic part.

The radical constituting the carbon-based chain of said flame retardant may or may not be substituted by one or more substituents other than bromine atoms. The substituent(s) can be selected from —OH, halogen other than Br, —NR^{a}C(O)R^{b}, —C(O)NR^{a}R^{b}, —CN, —NO₂, —NR^{a}R^{b}, —OR^{a}, —SR^{a}, —CO₂R^{a}, —OC(O)OR^{a}, —C(O)H or —C(O)R^{a}, in which R^{a} and R^{b} are, independently of one another, hydrogen, unsubstituted C₁-C₂₀ alkyl, unsubstituted C₂-C₂₀ alkenyl, unsubstituted C₂-C₂₀ alkynyl, unsubstituted C₃-C₂₀ cycloalkyl, unsubstituted C₃-C₂₀ cycloalkenyl or unsubstituted C₆-C₁₈ aryl.

The term “ether” refers to compounds of formula R^{c}—O—R^{d} in which R^{c} and R^{d} are, independently of each other, a C₃-C₂₀ alkyl, a C₃-C₂₀ alkenyl, a C₃-C₂₀ alkynyl, a C₃-C₂₀ cycloalkyl, a C₃-C₂₀ cycloalkenyl or a C₆-C₁₈ aryl and may or may not be substituted by one or more —OH, halogen, —NR^{a}C(O)R^{b}, —C(O)NR^{a}R^{b}, —CN, —NO₂, —NR^{a}R^{b}, —OR^{a}, —SR^{a}, —CO₂R^{a}, —OC(O)OR^{a}, —C(O)H or —C(O)R^{a} substituents, in which R^{a} and R^{b} are as defined above.

Depending on the length of its carbon-based chain, said flame retardant can comprise from 2 to 15 bromine atoms, advantageously from 2 to 14 bromine atoms, preferably from 2 to 13 bromine atoms, more preferentially from 2 to 12 bromine atoms, in particular from 2 to 11 bromine atoms, more particularly from 2 to 10 bromine atoms.

Favorably, said flame retardant A is selected from the compounds A-1, A-2, A-3, A-4, A-5, A-6, A-7, A-8 and A-9

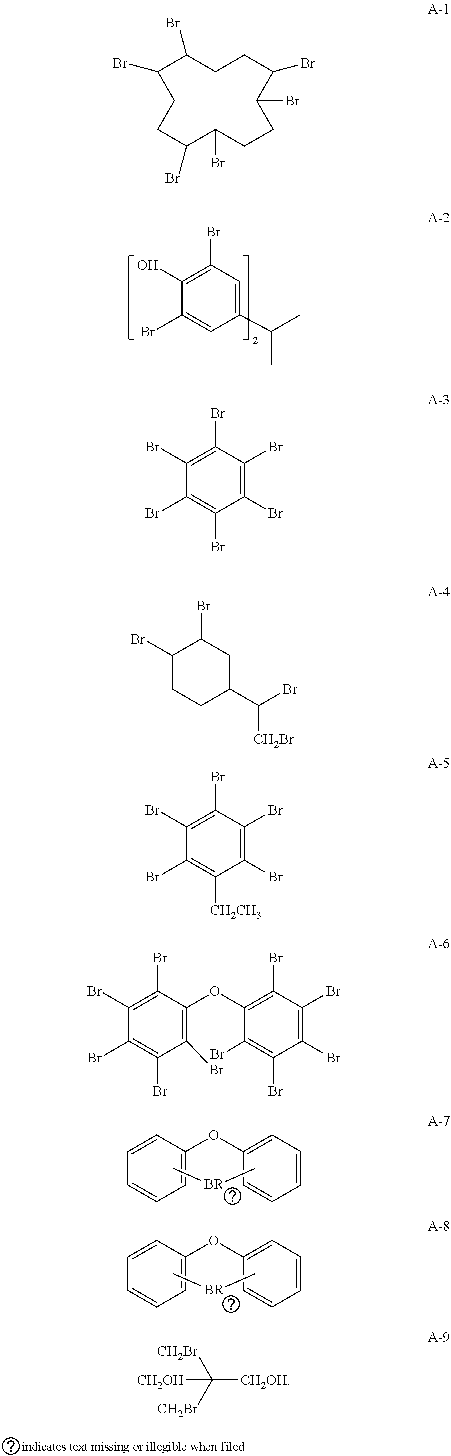

More particularly favorably, said flame retardant A is selected from the compounds A-1, A-2 and A-6.

Preferably, when the plastic consists of a thermosetting polymer as defined above, said flame retardant A is the compound A-2.

Alternatively, when the plastic consists of a thermoplastic polymer as defined above, said flame retardant A is the compound A-1 or A-6.

Preferably, when the plastic consists of a thermoplastic polymer selected from the group consisting of poly(methyl methacrylate), acrylonitrile/butadiene/styrene (ABS), polyamide, polycarbonate, polyetheretherketone, polyethylene, polypropylene, polystyrene, poly(vinyl chloride), polyvinylidene fluoride (PVDF) and polytetrafluoroethylene (PTFE), said flame retardant A is the compound A-1 or A-6. In particular, when the plastic consists of a thermoplastic polymer selected from the group consisting of polyethylene, polypropylene and polyamide, said flame retardant A is the compound A-6. In particular, when the plastic consists of a thermoplastic polymer selected from the group consisting of polystyrene and acrylonitrile/butadiene/styrene (ABS), said flame retardant A is the compound A-1.

### Stage b) of the Process

According to the present invention, the present process comprises a stage b) of recovery of a composition comprising said flame retardant A. Preferably, stage b) comprises a stage of depolymerization or of dissolution of said plastic in order to recover a composition comprising said flame retardant A. More particularly, stage b) comprises stages:

b1) depolymerization or dissolution of said plastic in order to form a mixture comprising said flame retardant A and residues from the plastic;
b2) purification of said mixture obtained in stage b1) in order to form said composition comprising said flame retardant A.

Stage b1)

The choice between a depolymerization and a dissolution depends on the type of plastics to be treated. A person skilled in the art, by virtue of his/her general knowledge, will know how to recognize the most suitable technique.

For example, when the plastic is a polyurethane or a polyester, stage b1) preferably employs a depolymerization stage. Said depolymerization can be carried out by alcoholysis, hydrolysis, ammonolysis or aminolysis reactions.

The alcoholysis reaction comprises bringing the plastic and an alcohol into contact. Preferably, the alcohol has a boiling point of between 30° C. and 350° C., advantageously between 50° C. and 325° C., preferably between 60° C. and 300° C. Preferably, the alcohol is selected from glycerol, methanol or a glycol compound. Preferably, said glycol compound has a molecular weight of less than 1000 g/mol, in particular of less than 500 g/mol. According to a preferred embodiment, the glycol compound is selected from the group consisting of ethylene glycol, diethylene glycol, triethylene glycol, tetraethylene glycol, 1,3-propylene glycol, 1,2-propylene glycol, di-, tri- and tetra(1,2-propylene glycol), butanediol and hexanediol. The molar ratio of the alcohol to the —[R¹—NH—CO—O—R²]— unit is between 1/1 and 5/1, advantageously between 1/1 and 4/1, preferably between 1/1 and 3/1, in particular between 1/1 and 2/1. Said alcoholysis reaction can be carried out in the presence of an amine of formula R³ₙNH₃₋ₙ in which R³ is an alkyl radical comprising from 1 to 10 carbon atoms, optionally substituted by one or more OH groups or one or more NH₂ groups; and n is 1 or 2. If the amine comprises several R³ groups as defined above, these can be identical or different or linked together to form a heterocycle. Preferably, said amine is of formula R³ₙNH₃₋ₙ in which R³ is an alkyl radical comprising from 1 to 4 carbon atoms substituted by one or more OH groups or one or more NH₂ groups; n being as defined above. In particular, said amine can be a compound, the molecular weight of which is less than 200 g/mol, preferably less than 150 g/mol. More particularly, said amine can be chosen from methanolamine, ethanolamine, diethanolamine, dipropanolamine and triethanolamine, and the combinations of these. Alternatively, said amine can be HMTA (i.e. hexamethylenetetramine) or diamines, such as ethylenediamine or hexamethylenediamine. The alcoholysis reaction can also be carried out in the presence of a catalyst. Said catalyst can be an acetate of an alkali metal, such as lithium acetate, sodium acetate or potassium acetate. The amount of catalyst can be between 0.01% and 5%, with respect to the weight of polyurethane, advantageously between 0.1% and 5% by weight, preferably between 0.5% and 5% by weight and more preferentially between 1% and 5% by weight. Carrying out the depolymerization by alcoholysis results in the formation of polyol compounds and possibly of urethane compounds.

The hydrolysis reaction comprises bringing the plastic and water into contact. The stage of depolymerization of polyurethane by hydrolysis is preferably carried out at high pressure, advantageously at a pressure of greater than 2 bars absolute, preferably of greater than 5 bars absolute, in particular of greater than 10 bars absolute. The stage of depolymerization of polyurethane by hydrolysis is preferably carried out at a temperature of from 100° C. to 300° C., preferably from 150° C. to 300° C. Thus, the water is at least partly in the form of steam. The reaction time for this hydrolysis stage is from 1 minute to 5 hours, preferably from 5 minutes to 2 hours, in particular from 10 minutes to 1 hour. Carrying out the depolymerization by hydrolysis results in the formation of polyol compounds, amine compounds and CO₂.

The stage of depolymerization of the plastic can be carried out in the presence of NH₃ (ammonolysis reaction), of a primary amine or of a secondary amine (aminolysis reaction). The primary or secondary amine is preferably of formula R⁴R⁵R⁶N in which R⁴, R⁵ and R⁶ are, independently of one another, selected from H, C₁-C₁₀ alkyl which is unsubstituted or substituted by an NH₂ group and C₁-C₁₀ alkenyl which is unsubstituted or substituted by an NH₂ group, preferably H, C₁-C₅ alkyl which is unsubstituted or substituted by an NH₂ group and C₁-C₅ alkenyl which is unsubstituted or substituted by an NH₂ group; provided that at least one of the R⁴, R⁵ and R⁶ groups represents a hydrogen and that R⁴, R⁵ and R⁶ are not simultaneously a hydrogen. In particular, the amine can be diethylenetriamine or triethylenetetramine. This stage can be carried out at a temperature of between 50° C. and 300° C., preferably between 100° C. and 250° C. According to another embodiment, when the plastic consists of polystyrene, stage b1) is carried out by bringing said plastic into contact with an organic solvent S1 capable of dissolving said plastic. Preferably, this dissolution stage can be carried out at a temperature ranging from 10° C. to 150° C., preferably from 20° C. to 100° C. According to a preferred embodiment, said organic solvent 51 is selected from the group consisting of n-octane, n-dodecane, cyclohexane, methylcyclohexane, benzene, toluene, naphthalene, styrene, o-xylene, ethylbenzene, p-diethylbenzene, p-cymene, chloromethane, 1,1-dichloroethylene, ethylene dichloride, chloroform, 1,1-dichloroethane, trichloroethylene, carbon tetrachloride, chlorobenzene, o-dichlorobenzene, tetrahydrofuran, 1,4-dioxane, dibenzyl ether, acetone, methyl ethyl ketone, cyclohexanone, diethyl ketone, acetophenone, methyl isobutyl ketone, methyl isoamyl ketone, isophorone, di(isobutyl) ketone, methyl acetate, ethyl formate, 1,2-propylene carbonate, ethyl acetate, diethyl carbonate, n-butyl acetate, 2-ethoxyethyl acetate, isoamyl acetate, 2-nitropropane, nitrobenzene, ethylenediamine, pyridine, morpholine, aniline, N-methyl-2-pyrrolidone, cyclohexylamine, quinoline, N,N-dimethylformamide, carbon disulfide, dimethyl sulfoxide, ethanediol, ethanol, allyl alcohol, 1-propanol, 2-propanol, 1-butanol, 2-butanol, benzyl alcohol, cyclohexanol, diacetone alcohol, ethylene glycol monoethyl ether, diethylene glycol monomethyl ether, diethylene glycol monoethyl ether, ethylene glycol monobutyl ether, diethylene glycol monobutyl ether, 1-decanol, benzoic acid, stearic acid, phenol, resorcinol, m-cresol, methyl salicylate, ethylene glycol, glycerol and propylene glycol, or a mixture of these. This embodiment by dissolution is favored; however, an alternative embodiment by depolymerization, below, can be envisaged

According to another alternative embodiment, when the plastic consists of polystyrene, stage b1) is carried out by thermal depolymerization. Thus, said plastic is preferably heated to a temperature of between 200° C. and 500° C. At such a temperature, the polystyrene is in the molten state and then depolymerizes. According to a specific embodiment, the depolymerization can be carried out in the presence of a radical initiator, advantageously a radical initiator of peroxide type. Preferably, the radical initiator is selected from the group consisting of an organic peroxide, an inorganic peroxide or superoxide, such as barium peroxide (BaO₂), potassium superoxide (KO₂) or cesium superoxide (CsO₂), a percarbonate, a peroxyhydrate compound, their salts and also their mixture. Mention may be made, as catalyst for initiation of the depolymerization, of hydrogen peroxide (H₂O₂), azobisisobutyronitrile (AIBN), sodium carbonate peroxyhydrate (2Na₂CO₃.3H₂O₂), or potassium or magnesium or calcium carbonate peroxyhydrate, ammonium carbonate peroxyhydrate ((NH₄)₂CO₃.H₂O₂), urea hydrogen peroxide (CO(NH₂)₂.H₂O₂), sodium sulfate peroxyhydrate (2Na₂SO₄.H₂O₂.2H₂O), complexes of H₂O₂ and of inorganic salts, poly(vinylpyrrolidone) polymer peroxyhydrate (PVP.H₂O₂), persulfates, permanganates, perborates or phosphate salt peroxyhydrates.

According to another embodiment, when the plastic consists of poly(methyl methacrylate), stage b1) can be carried out by thermal depolymerization, preferably at a temperature of from 200° C. to 500° C. At such a temperature, the poly(methyl methacrylate) is in the solid or molten state. According to a specific embodiment, the depolymerization can be carried out in the presence of a radical initiator, advantageously a radical initiator of peroxide type. Preferably, the radical initiator is selected from the group consisting of an organic peroxide, an inorganic peroxide or superoxide, such as barium peroxide (BaO₂), potassium superoxide (KO₂) or cesium superoxide (CsO₂), a percarbonate, a peroxyhydrate compound, their salts and also their mixture. Mention may be made, as catalyst for initiation of the depolymerization, of hydrogen peroxide (H₂O₂), azobisisobutyronitrile (AIBN), sodium carbonate peroxyhydrate (2Na₂CO₃.3H₂O₂), or potassium or magnesium or calcium carbonate peroxyhydrate, ammonium carbonate peroxyhydrate ((NH₄)₂CO₃.H₂O₂), urea hydrogen peroxide (CO(NH₂)₂.H₂O₂), sodium sulfate peroxyhydrate (2Na₂SO₄.H₂O₂.2H₂O), complexes of H₂O₂ and of inorganic salts, poly(vinylpyrrolidone) polymer peroxyhydrate (PVP.H₂O₂), persulfates, permanganates, perborates or phosphate salt peroxyhydrates.

Alternatively, when the plastic consists of poly(methyl methacrylate), stage b1) can be carried out by dissolving the latter in an organic solvent S2 capable of dissolving poly(methyl methacrylate). Preferably, said organic solvent S2 is methyl methacrylate. The use of methyl methacrylate to dissolve poly(methyl methacrylate) is advantageous because the liquid phase thus formed can be used, after an optional filtration stage, in a process for the production of poly(methyl methacrylate). Alternatively, said organic solvent S2 can be selected from the group consisting of toluene, acetone, butanone, cyclohexanone, nitroethane, chloroform, dichloromethane, benzene, chlorobenzene, xylene, methoxybenzene, diethyl phthalate, methoxypropyl acetate, ethyl acetate, ethyl lactate and formic acid.

Preferably, the dissolution of the poly(methyl methacrylate) is carried out at a temperature of from 20° C. to 200° C., preferably from 25° C. to 100° C., preferably from 30° C. to 80° C. The dissolution can be carried out under pressure.

According to another embodiment, when said plastic consists of polyhydroxyalkanoate (i.e. PHA), stage b1) is carried out by depolymerization. Preferably, the depolymerization is carried out by hydrolysis or thermally. The thermal depolymerization is carried out at a temperature of greater than 100° C., advantageously of greater than 150° C., preferably of greater than 200° C.; and of less than 400° C., preferably of less than 300° C. Preferably, the thermal depolymerization is carried out in the presence of an inert gas, such as nitrogen, CO₂ or argon. Preferably, the thermal depolymerization is carried out at a pressure of less than 1 bar absolute. Alternatively, the thermal depolymerization can be carried out under partial vacuum. Preferably, the thermal depolymerization is carried out in the presence of a depolymerization catalyst. Preferably, said depolymerization catalyst is selected from the group consisting of acid catalysts, such as mixed oxides, zeolites, aluminas, titanium or zirconium oxides doped with one or more of the elements P, S, W, B, Nb or Ta. Alternatively, stage b1) can be carried out by hydrolysis. In this case, stage b1) is carried out at a temperature of from 20° C. to 100° C. in the presence of water. The depolymerization of PHA by hydrolysis is to be favored in comparison with the thermal route.

According to another embodiment, when the plastic consists of polylactic acid, stage b) or b1) can be carried out by dissolution or depolymerization. The dissolution of the polylactic acid can be carried out in the presence of an organic solvent selected from tetrahydrofuran, dioxane, dioxolane, m-cresol, pyridine, N-methylpyrrolidone, butyrolactone, ethyl acetate, propylene carbonate, acetone, acetonitrile, nitrobenzene, dimethylacetamide, dichloromethane or chloroform. Alternatively, the dissolution of the polylactic acid can be carried out in the presence of an organic solvent such as a lactic acid ester. The lactic acid ester can be methyl lactate, ethyl lactate, isopropyl lactate, butyl lactate or hexyl lactate. The dissolution stage can be followed by a filtration stage in order to remove any particles which are not soluble in the organic solvent. The depolymerization of the polylactic acid can be carried out by hydrolysis at a temperature of from 80° C. to 180° C., preferably from 100° C. to 150° C., in particular from 120° C. to 140° C. The depolymerization by hydrolysis can be carried out at a pressure of less than 1 bara or at a pressure of from 1 bara to 10 bara. The depolymerization by hydrolysis can be carried out in the presence of water or of an alkaline solution of NaOH or KOH. The depolymerization by hydrolysis can optionally be carried out in the presence of a catalyst such as a Lewis acid chosen from tin octoate, tin lactate, antimony octoate, zinc octoate, APTS or triazabicyclodecene. The depolymerization by hydrolysis can optionally be carried out in the presence of a catalyst such as a Brønsted acid.

According to another embodiment, when said plastic consists of a polyolefin, such as polyethylene or polypropylene, stage b1) is carried out by dissolution in an organic solvent S3. Stage b1) can be carried out at a temperature of from 100° C. to 300° C. Said organic solvent S3 can be dimethylformamide, dimethyl sulfoxide, xylenes, tetralin, decalin or 1,2,4-trichlorobenzene.

According to another embodiment, when said plastic consists of polyimide, of polycarbonate or of poly(ether imide), stage b1) is carried out by depolymerization as described above for polyurethane.

According to another embodiment, when said plastic consists of poly(methacryl imide), stage b1) is carried out as described above for poly(methyl methacrylate).

According to another embodiment, when said plastic consists of poly(vinyl chloride) or poly(ethylene/vinyl acetate), stage b1) is carried out as described above for a polyolefin.

Whatever the plastic and the way in which stage b1) is carried out, a mixture comprising said flame retardant A and residues from the plastic is generally obtained during this stage. Residues from the plastic is understood to mean both the dissolved plastic and monomeric or polymeric fragments of the plastic or other coproducts resulting from the depolymerization of the plastic.

Stage b2)

Stage b2) consists of the purification of the mixture obtained in stage b1) in order to form said composition comprising said flame retardant A. The purification stage can contain a distillation, a filtration, a precipitation under cold conditions or by contact with a nonsolvent, a liquid-liquid extraction, a solid-liquid extraction, a purification over membrane or over zeolite, molecular sieve or activated carbon, or a combination of these. A person skilled in the art, by virtue of his/her general knowledge, will determine the most appropriate purification technique. The purification over zeolite or molecular sieve or activated carbon can be used to remove, for example, residues of solvents.

For example, when the flame retardant A is the compound A-1, A-2 or A-6, stage b2) can be carried out by precipitation of the flame retardant A in the presence of water, in view of the low solubility of this in water. When the flame retardant A is the compound A-6, stage b2) can also be carried out by precipitation of the flame retardant A in the presence of acetone or of toluene. The purification stage can also comprise one or more washing and/or drying stages. Said composition comprising said flame retardant A obtained in stage b2) can be in solid form or in liquid form.

Preferably, when said composition comprising said flame retardant A is in liquid form, the content by weight of flame retardant A in said composition is greater than 30%, advantageously greater than 40%, preferably greater than 50%, more preferentially greater than 60%, in particular greater than 70%, more particularly greater than 80%, favorably greater than 90%, more favorably greater than 95%.

### Stage c)

According to the present process, stage c) consists in bringing said composition comprising said flame retardant A into contact, in an organic solvent S4, with a compound A′ selected from the group consisting of Na₂S₂, K₂S₂, a potassium or sodium alkyl xanthate, and a compound of formula Y—SH in which Y is an alkali metal, an alkaline earth metal, a metal selected from rare earth metals, a —C(NH₂)═NH unit or a —C(NR¹R²)=NR³ unit with R¹, R² and R³ being, independently of one another, H or C₁-C₁₀ alkyl, provided that R¹, R² and R³ are not simultaneously H; in order to form a mixture C comprising a compound B consisting of a carbon-based chain substituted by at least two —SH functional groups. The reaction between the flame retardant A and the compound A′ makes possible the replacement of all or part of the bromine atoms by an —SH functional group. Thus, the structure of the carbon-based chain of the flame retardant A, i.e. the number of carbon atoms and its structure (alkyl, aryl, cycloalkyl, and the like), is not modified by the implementation of stage c). The compound B obtained during this stage will thus have an identical carbon-based chain to that of the flame retardant A.

The organic solvent S4 is DMSO or an alcohol of formula R—OH in which R is a linear or branched alkyl comprising from 1 to 20 carbon atoms. Preferably, the organic solvent S4 is selected from the group consisting of methanol, ethanol, propanol, isopropanol, butanol, sec-butanol, isobutanol, tert-butanol, pentan-1-ol, 3-methylbutan-1-ol, 2-methylbutan-1-ol, 2,2-dimethylpropan-1-ol, pentan-3-ol, pentan-2-ol, 3-methylbutan-2-ol, 2-methylbutan-2-ol, hexan-1-ol, hexan-2-ol, hexan-3-ol, 2-methylpentan-1-ol, 3-methylpentan-1-ol, 4-methylpentan-1-ol, 2-methylpentan-2-ol, 3-methylpentan-2-ol, 4-methylpentan-2-ol, 2-methylpentan-3-ol, 3-methylpentan-3-ol, 2,2-dimethylbutan-1-ol, 2,3-dimethylbutan-1-ol, 3,3-dimethylbutan-1-ol, 2,3-dimethylbutan-2-ol, 3,3-dimethylbutan-2-ol, 2-ethylbutan-1-ol, heptan-1-ol, heptan-2-ol, heptan-3-ol, heptan-4-ol, 2-methylhexan-1-ol, 3-methylhexan-1-ol, 4-methylhexan-1-ol, 5-methylhexan-1-ol, 6-methylhexan-1-ol, 2-methylhexan-2-ol, 3-methylhexan-2-ol, 4-methylhexan-2-ol, 5-methylhexan-2-ol, 6-methylhexan-2-ol, 2-methylhexan-3-ol, 3-methylhexan-3-ol, 4-methylhexan-3-ol, 5-methylhexan-3-ol, 6-methylhexan-3-ol, 2-ethylpentan-1-ol, 3-ethylpentan-1-ol, 3-ethylpentan-2-ol, 2-ethylpentan-3-ol and 3-ethylpentan-3-ol. The organic solvent 54 can also be ethylene glycol or glycerol. In particular, the organic solvent 54 is methanol.

Preferably, the compound A′ is a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl, provided that R¹, R² and R³ are not simultaneously H. The alkali metal can be Li, Na, K or Cs. The alkaline earth metal can be Mg, Ca or Ba. Mention may in particular be made, as rare earth metal, of lanthanum, cerium, praseodymium, neodymium, promethium, samarium, europium, gadolinium, terbium, dysprosium, holmium, erbium, thulium, ytterbium, lutetium, yttrium and scandium.

Preferably, the compound A′ is a compound of formula Y—SH in which Y is an alkali metal.

Preferably, the compound A′ is selected from the group consisting of LiSH, NaSH and KSH. When the compound A′ is selected from the group consisting of LiSH, NaSH and KSH, stage c) also results in the formation respectively of LiBr, NaBr and KBr. The compound NaBr can be upgraded in various processes, including that of the production of bromine Br₂. It is thus particularly advantageous to use NaSH as compound A′ in the present process.

Preferably, stage c) is carried out in the presence of H₂S. Preferably, the molar ratio H₂S/A′ is less than 0.4, more preferentially less than 0.3. In particular, the molar ratio H₂S/A′ is from 0.05 to 0.4, more particularly from 0.1 to 0.3. In a preferred embodiment, the compound A′ is NaSH and the molar ratio H₂S/NaSH is from 0.05 to 0.4, more particularly from 0.1 to 0.3.

The presence of an oxidizing agent, in particular oxygen, during stage c) can result in the formation of impurities, such as disulfide compounds. Thus, stage c) is carried out with an oxygen content of less than 1000 ppm, preferably of less than 500 ppm, more preferentially of less than 100 ppm, in particular of less than 10 ppm. Preferably, stage c) is carried out in the absence of oxygen. Thus, in order to avoid the presence of oxygen in the reactor in which stage c) is being carried out, a gas stream comprising an inert gas is introduced into the reactor prior to this stage c). Generally, the compound A and the organic solvent 54 are introduced into the reactor and then the reactor is purged with the gas stream comprising an inert gas. Once this purging stage has been carried out, the compound A′ is introduced into the reactor, optionally with H₂S. Preferably, the inert gas is nitrogen.

When the compound A′ is NaSH or KSH, this can be prepared in situ in the reactor in which stage c) is being carried out. Alternatively, NaSH or KSH can be prepared in another reactor and then introduced into the reactor in which stage c) is being carried out in the form of an aqueous or nonaqueous solution.

Preferably, NaSH is prepared by reaction between NaOH and H₂S. Preferably, KSH is prepared by reaction between KOH and H₂S.

Preferably, stage c) is carried out at a temperature of from 20° C. to 150° C., preferably from 20° C. to 100° C. Preferably, stage c) is carried out at a pressure of from 1 bar absolute to 15 bars absolute, more preferentially from 1 bar absolute to 7 bars absolute, in particular from 2 to 3 bars absolute. Thus, stage c) is carried out at a temperature of from 20° C. to 150° C., preferably from 20° C. to 100° C.; and at a pressure of from 1 bar to 15 bars, preferably from 1 bar to 7 bars and in particular from 2 bars to 3 bars.

The temperature can increase as stage c) is carried out. Thus, the temperature can increase stepwise while being within the abovementioned range.

Preferably, stage c) is carried out for a period of time of from 1 to 100 hours, preferably from 2 to 24 hours.

Preferably, the compound A′ is introduced at stoichiometry or in excess with respect to the replacement number for bromine atoms which is desired on the compound A.

The compound B formed during this stage c) consists of at least two —SH functional groups, that is to say that at least two of the bromine atoms of the compound A have been replaced by an —SH functional group.

The other R substituents are, independently of one another, —SH, —Br, —OH, —H, ═S or form a C═C double bond, by a reaction for elimination of H—R with R═OH or Br, between the carbon atom carrying the R substituent and a carbon atom adjacent to the latter carrying a hydrogen atom; preferably, the other R substituents are, independently of one another, —SH, —Br, —OH or form a C═C double bond, by a reaction for elimination of H—R with R═OH or Br, between the carbon atom carrying the R substituent and a carbon atom adjacent to the latter carrying a hydrogen atom;

more preferentially, the other R substituents are, independently of one another, —SH, —OH or form a C═C double bond, by a reaction for elimination of H—R with R═OH or Br, between the carbon atom carrying the R substituent and a carbon atom adjacent to the latter carrying a hydrogen atom;
in particular, the other R substituents are, independently of one another, —SH or —OH.

If, in a compound B, the OH group is carried by an unsaturated carbon atom, that is to say a carbon atom forming a C═C double bond with another carbon atom, then, by isomerization, the R substituent can be ═O, that is to say form a carbonyl functional group with the carbon atom.

According to a preferred embodiment, said compound B is selected from the group consisting of the compounds B-1, B-2, B-3, B-4, B-5, B-6, B-7, B-8 and B-9

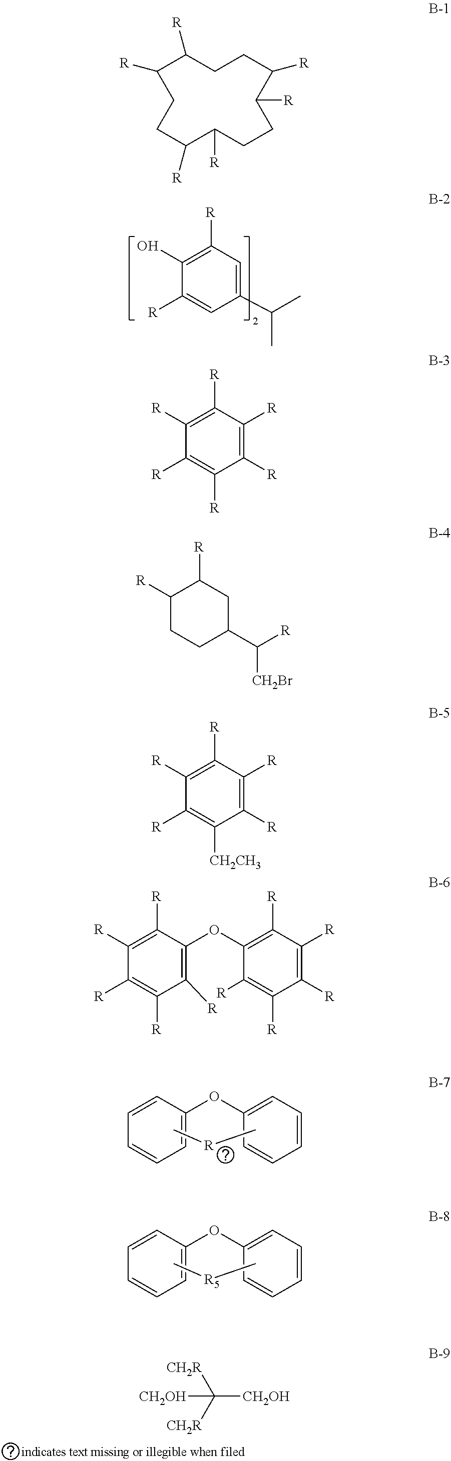

in which at least two R substituents are —SH; the other R substituents are, independently of one another, —SH, —Br, —OH, —H, ═S or form a C═C double bond, by a reaction for elimination of H—R with R═OH or Br, between the carbon atom carrying the R substituent and a carbon atom adjacent to the latter carrying a hydrogen atom; preferably, the other R substituents are, independently of one another, —SH, —Br, —OH or form a C═C double bond, by a reaction for elimination of H—R with R═OH or Br, between the carbon atom carrying the R substituent and a carbon atom adjacent to the latter carrying a hydrogen atom; more preferentially, the other R substituents are, independently of one another, —SH, —OH or form a C═C double bond, by a reaction for elimination of H—R with R═OH or Br, between the carbon atom carrying the R substituent and a carbon atom adjacent to the latter carrying a hydrogen atom; in particular, the other R substituents are, independently of one another, —SH or —OH.

As mentioned above, the nature of the R substituent will also depend on the nature of the compound B. Thus, when the R substituents are carried by an aryl group, the R substituent cannot be —H or ═S or form a C═C double bond. In this case, the R substituents are selected from —SH, —Br or —OH, preferably —SH or —OH. Thus, in the compounds B-2, B-3, B-5, B-6, B-7 and B-8, the R substituents are, independently of one another, selected from —SH, —Br or —OH, preferably —SH or —OH.

If appropriate, stage c) can also be carried out in the presence of NH₃/NH₄OH or an alkylamine.

This makes it possible to limit the formation of impurities, such as sulfides.

Depending on the temperature at which stage c) is carried out, the temperature and the pressure can be decreased before carrying out stage d).

If the compound B still contains bromine atoms after carrying out stage c), an optional stage c′) of bringing the compound B obtained after stage c) into contact with an alkali metal or alkaline earth metal hydroxide can be carried out. This stage can be carried out at a temperature of from 20° C. to 100° C. and at a pressure of from 1 bar absolute to 15 bars absolute. The alkali metal or alkaline earth metal hydroxide is preferably NaOH, KOH, Ca(OH)₂ or Mg(OH)₂, in particular NaOH. Via this stage c′), the bromine atoms possibly present on the compound B are replaced by an —OH functional group. If the compound A′ is NaSH, it is preferable to use NaOH in order to also form NaBr.

Stage c′) can be simultaneous with stage c). In this case, stage c) comprises bringing said flame retardant A into contact, in an organic solvent, with the compound A′, preferably NaSH, in the presence of an alkali metal hydroxide, preferably NaOH, and optionally in the presence of H₂S, in order to form the mixture C comprising said compound B.

Thus, the compound B can contain at least two —SH functional groups and at least one —OH functional group and optionally at least one bromine atom or one C═C double bond, by a reaction for elimination of H—R with R═OH or Br, between the carbon atom carrying the R substituent and a carbon atom adjacent to the latter carrying a hydrogen atom. Preferably, the compound B can contain at least two —SH functional groups and at least one —OH functional group and optionally at least one C═C double bond, by a reaction for elimination of H—R with R═OH or Br, between the carbon atom carrying the R substituent and a carbon atom adjacent to the latter carrying a hydrogen atom, and is devoid of bromine atoms.

In particular, the compound B is selected from the group consisting of the compounds B-1, B-2, B-3, B-4, B-5, B-6, B-7, B-8 and B-9 as defined above in which each R substituent is independently selected from —SH, —OH, —Br or forms a C═C double bond, by a reaction for elimination of H—R with R═OH or Br, between the carbon atom carrying the R substituent and a carbon atom adjacent to the latter carrying a hydrogen atom; provided that at least two R substituents are —SH.

More particularly, the compound B is selected from the group consisting of the compounds B-1, B-2, B-3, B-4, B-5, B-6, B-7, B-8 and B-9 as defined above in which each R substituent is independently selected from —SH, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;

According to a preferred embodiment, the compound A is the compound A-1 and the compound B is the compound B-1 in which at least two R substituents are —SH; preferably, the compound B is the compound B-1 in which at least three R substituents are —SH; more preferentially, the compound B is the compound B-1 in which at least four R substituents are —SH; in particular, the compound B is the compound B-1 in which at least five R substituents are —SH; more particularly, the compound B is the compound B-1 in which all the R substituents are —SH.

In particular, the compound B-1 is any one of the compounds shown below in table 1a; more particularly, the compound B-1 is any one of the compounds (devoid of bromine atoms) shown below in table 1b.

TABLE 1a

| R | R | R | R | R | R |
|---|---|---|---|---|---|
| SH | SH | Br | Br | Br | Br |
| SH | SH | SH | Br | Br | Br |
| SH | SH | SH | SH | Br | Br |
| SH | SH | SH | SH | SH | Br |
| SH | SH | SH | SH | SH | SH |
| SH | SH | Br | Br | Br | OH |
| SH | SH | SH | Br | Br | OH |
| SH | SH | SH | SH | Br | OH |
| SH | SH | SH | SH | SH | OH |
| SH | SH | Br | Br | OH | OH |
| SH | SH | SH | Br | OH | OH |
| SH | SH | SH | SH | OH | OH |
| SH | SH | Br | OH | OH | OH |
| SH | SH | SH | OH | OH | OH |
| SH | SH | OH | OH | OH | OH |
| SH | SH | Br | Br | Br | C═ |
| SH | SH | SH | Br | Br | C═ |
| SH | SH | SH | SH | SH | C═ |
| SH | SH | Br | Br | C═ | C═ |
| SH | SH | SH | Br | C═ | C═ |
| SH | SH | SH | SH | C═ | C═ |
| SH | SH | Br | C═ | C═ | C═ |
| SH | SH | SH | C═ | C═ | C═ |
| SH | SH | C═ | C═ | C═ | C═ |
| SH | SH | OH | OH | OH | C═ |
| SH | SH | OH | OH | C═ | C═ |
| SH | SH | OH | C═ | C═ | C═ |
| SH | SH | SH | OH | OH | C═ |
| SH | SH | SH | OH | C═ | C═ |
| SH | SH | SH | SH | OH | C═ |
| SH | SH | Br | Br | OH | C═ |
| SH | SH | Br | OH | OH | C═ |
| SH | SH | Br | OH | C═ | C═ |
| SH | SH | SH | Br | OH | C═ |
| SH | SH | SH | OH | OH | C═ |
| SH | SH | SH | OH | C═ | C═ |
| SH | SH | SH | SH | OH | C═ |

Each line represents a compound B-1 in which the R substituents are as mentioned in the line independently of their position. For example, the final line of table 1a describes a compound B-1 in which four R substituents are —SH, one R substituent is —OH and one R substituent is C═. The term “C═” means that the carbon atom carrying the R substituent forms a carbon—carbon double bond with an adjacent carbon atom which carried a hydrogen atom as indicated in the present patent application, the double bond being obtained by elimination of H—R.

TABLE 1b

| R | R | R | R | R | R |
|---|---|---|---|---|---|
| SH | SH | SH | SH | SH | SH |
| SH | SH | SH | SH | SH | OH |
| SH | SH | SH | SH | OH | OH |
| SH | SH | SH | OH | OH | OH |
| SH | SH | OH | OH | OH | OH |
| SH | SH | SH | SH | SH | C═ |
| SH | SH | SH | SH | C═ | C═ |
| SH | SH | SH | C═ | C═ | C═ |
| SH | SH | C═ | C═ | C═ | C═ |
| SH | SH | OH | OH | OH | C═ |
| SH | SH | OH | OH | C═ | C═ |
| SH | SH | OH | C═ | C═ | C═ |
| SH | SH | SH | OH | OH | C═ |
| SH | SH | SH | OH | C═ | C═ |
| SH | SH | SH | SH | OH | C═ |
| SH | SH | SH | OH | OH | C═ |
| SH | SH | SH | OH | C═ | C═ |
| SH | SH | SH | SH | OH | C═ |

Favorably, the compound B-1 is such that two R substituents are —SH and four R substituents are —OH; or such that three R substituents are —SH and three R substituents are —OH; or such that four R substituents are —SH and two R substituents are —OH; or such that five R substituents are —SH and one R substituent is —OH or such that six R substituents are —SH.

According to another embodiment, the compound A is the compound A-2 and the compound B is the compound B-2 in which at least two R substituents are —SH; preferably, the compound B is the compound B-2 in which at least three R substituents are —SH; more preferentially, the compound B is the compound B-2 in which all the R substituents are —SH.

In particular, the compound B-2 is any one of the compounds shown below in table 2:

TABLE 2

| R | R | R | R |
|---|---|---|---|
| SH | SH | Br | Br |
| SH | SH | SH | Br |
| SH | SH | SH | SH |
| SH | SH | Br | OH |
| SH | SH | SH | OH |
| SH | SH | OH | OH |

Each line represents a series of compounds B-2 in which the R substituents are as mentioned in the line independently of their position. For example, the final line of table 2 describes a compound B-2 in which two R substituents are —SH and two R substituents are —OH.

Favorably, the compound B-2 is such that two R substituents are —SH and two R substituents are —OH; or such that three R substituents are —SH and one R substituent is —OH; or such that four R substituents are —SH.

According to a preferred embodiment, the compound A is the compound A-6 and the compound B is the compound B-6 in which at least two R substituents are —SH; preferably, the compound B is the compound B-6 in which at least three R substituents are —SH; more preferentially, the compound B is the compound B-6 in which at least four R substituents are —SH; in particular, the compound B is the compound B-6 in which at least five R substituents are —SH; more particularly, the compound B is the compound B-6 in which at least six R substituents are —SH; favorably, the compound B is the compound B-6 in which at least seven R substituents are —SH; advantageously favorably, the compound B is the compound B-6 in which at least eight R substituents are —SH; preferentially favorably, the compound B is the compound B-6 in which at least nine R substituents are —SH; particularly favorably, the compound B is the compound B-6 in which all the R substituents are —SH.

In particular, the compound B-6 is any one of the compounds shown below in table 3:

TABLE 3

| R | R | R | R | R | R | R | R | R | R |
|---|---|---|---|---|---|---|---|---|---|
| SH | SH | Br | Br | Br | Br | Br | Br | Br | Br |
| SH | SH | SH | Br | Br | Br | Br | Br | Br | Br |
| SH | SH | SH | SH | Br | Br | Br | Br | Br | Br |
| SH | SH | SH | SH | SH | Br | Br | Br | Br | Br |
| SH | SH | SH | SH | SH | SH | Br | Br | Br | Br |
| SH | SH | SH | SH | SH | SH | SH | Br | Br | Br |
| SH | SH | SH | SH | SH | SH | SH | SH | Br | Br |
| SH | SH | SH | SH | SH | SH | SH | SH | SH | Br |
| SH | SH | SH | SH | SH | SH | SH | SH | SH | SH |
| SH | SH | Br | Br | Br | Br | Br | Br | Br | OH |
| SH | SH | SH | Br | Br | Br | Br | Br | Br | OH |
| SH | SH | SH | SH | Br | Br | Br | Br | Br | OH |
| SH | SH | SH | SH | SH | Br | Br | Br | Br | OH |
| SH | SH | SH | SH | SH | SH | Br | Br | Br | OH |
| SH | SH | SH | SH | SH | SH | SH | Br | Br | OH |
| SH | SH | SH | SH | SH | SH | SH | SH | Br | OH |
| SH | SH | SH | SH | SH | SH | SH | SH | SH | OH |
| SH | SH | Br | Br | Br | Br | Br | Br | OH | OH |
| SH | SH | SH | Br | Br | Br | Br | Br | OH | OH |
| SH | SH | SH | SH | Br | Br | Br | Br | OH | OH |
| SH | SH | SH | SH | SH | Br | Br | Br | OH | OH |
| SH | SH | SH | SH | SH | SH | Br | Br | OH | OH |
| SH | SH | SH | SH | SH | SH | SH | Br | OH | OH |
| SH | SH | SH | SH | SH | SH | SH | SH | OH | OH |
| SH | SH | Br | Br | Br | Br | Br | OH | OH | OH |
| SH | SH | SH | Br | Br | Br | Br | OH | OH | OH |
| SH | SH | SH | SH | Br | Br | Br | OH | OH | OH |
| SH | SH | SH | SH | SH | Br | Br | OH | OH | OH |
| SH | SH | SH | SH | SH | SH | Br | OH | OH | OH |
| SH | SH | SH | SH | SH | SH | SH | OH | OH | OH |
| SH | SH | Br | Br | Br | Br | OH | OH | OH | OH |
| SH | SH | SH | Br | Br | Br | OH | OH | OH | OH |
| SH | SH | SH | SH | Br | Br | OH | OH | OH | OH |
| SH | SH | SH | SH | SH | Br | OH | OH | OH | OH |
| SH | SH | SH | SH | SH | SH | OH | OH | OH | OH |
| SH | SH | Br | Br | Br | OH | OH | OH | OH | OH |
| SH | SH | SH | Br | Br | OH | OH | OH | OH | OH |
| SH | SH | SH | SH | Br | OH | OH | OH | OH | OH |
| SH | SH | SH | SH | SH | OH | OH | OH | OH | OH |
| SH | SH | Br | Br | OH | OH | OH | OH | OH | OH |
| SH | SH | SH | Br | OH | OH | OH | OH | OH | OH |
| SH | SH | SH | SH | OH | OH | OH | OH | OH | OH |
| SH | SH | Br | OH | OH | OH | OH | OH | OH | OH |
| SH | SH | SH | OH | OH | OH | OH | OH | OH | OH |
| SH | SH | OH | OH | OH | OH | OH | OH | OH | OH |

Each line represents a series of compounds B-6 in which the R substituents are as mentioned in the line independently of their position. For example, the final line of table 3 describes a compound B-6 in which two R substituents are —SH and eight R substituents are —OH.

Favorably, the compound B-6 is such that two R substituents are —SH and eight R substituents are —OH; or such that three R substituents are —SH and seven R substituents are —OH; or such that four R substituents are —SH and six R substituents are —OH; or such that five R substituents are —SH and five R substituents are —OH; or such that six R substituents are —SH and four R substituents are —OH; or such that seven R substituents are —SH and three R substituents are —OH; or such that eight R substituents are —SH and two R substituents are —OH; or such that nine R substituents are —SH and one R substituent is —OH; or such that ten R substituents are —SH.

Thus, stage c) results in the formation of a mixture C comprising said compound B, and optionally the unreacted compound A and the unreacted compound A′. Depending on the operating conditions, the mixture C can also contain H₂S, HBr, H₂O, solvent S4, residues related to the presence of NH₃/NH₄OH or of amines. The mixture C can also contain compounds B in which at least one R substituent is ═S or —H. The mixture C can also comprise byproducts, such as disulfide or sulfide compounds.

### Stage d)

Preferably, the mixture C obtained in stage c) or c′) is recovered and purified. The purification stage d) to be carried out depends on the products contained in the mixture C. Stage d) makes it possible to recover said purified compound B. The separation can be carried out by separation techniques conventional in the technical field, for example a distillation, crystallization, preparative chromatography, a filtration or a liquid-liquid extraction. The compound B can also be purified by conventional methods also well known to a person skilled in the art and for example chosen from purification over ion-exchange resins, filtration over activated carbon, diatomaceous earths or zeolites, chromatographic separation, crystallization, extraction, washing, precipitation and the like.

Preferably, stage d) comprise a stage of acidification of the mixture C. The acidification can, for example, be carried out with HCl or acetic acid. The mixture thus obtained can be washed, for example with the organic solvent 54 as defined above, then dried or filtered (for example over activated carbon) then dried.

Stage d) thus makes it possible to recover said compound B with a purity of greater than 90%, advantageously of greater than 92%, preferably of greater than 94%, more preferentially of greater than 96%, in particular of greater than 98%, more particularly of greater than 99%.

In particular, stage d) makes it possible to recover said compound B with a purity of greater than 90%, advantageously of greater than 92%, preferably of greater than 94%, more preferentially of greater than 96%, in particular of greater than 98%, more particularly of greater than 99%, said compound B being the compound B-1, B-2 or B-6 as described in the present patent application.

In the context of the present invention and of the present process, the preparation of compounds B devoid of bromine atoms is favored.

### Process for the Preparation of the Compound B

According to a second aspect, the present invention relates to the preparation of polythiol compounds, that is to say compounds comprising several, i.e. at least two, —SH functional groups on its carbon-based chain. In particular, the present invention relates to a process for the preparation of a compound B comprising at least two —SH functional groups, characterized in that it comprises bringing a compound A consisting of a carbon-based chain substituted by at least two bromine atoms into contact, in an organic solvent, with a compound A′ selected from the group consisting of Na₂S₂, K₂S₂, a potassium or sodium alkyl xanthate, and a compound of formula Y—SH in which Y is an alkali metal, an alkaline earth metal, a metal selected from rare earth metals, a —C(NH₂)═NH unit or a —C(NR¹R²)=NR³ unit with R¹, R² and R³ being, independently of one another, H or C₁-C₁₀ alkyl, provided that R¹, R² and R³ are not simultaneously H; said compound A being selected from the compounds A-1, A-2, A-3, A-4, A-5, A-6, A-7, A-8 and A-9 as defined above in order to form a compound B selected from the group consisting of the compounds B-1, B-2, B-3, B-4, B-5, B-6, B-7, B-8 and B-9 as defined above in which at least two R substituents are —SH.

Preferably, the compound A′ is a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl; more preferentially, the compound A′ is of formula Y—SH in which Y is an alkali metal, an alkaline earth metal or a metal selected from rare earth metals; in particular, the compound A′ is of formula Y—SH with Y an alkali metal.

The compound A′ can be selected from the group consisting of LiSH, NaSH and KSH. Thus, a compound selected from the group consisting of LiBr, NaBr and KBr is formed. According to a preferred embodiment, the compound A′ is NaSH and the compound NaBr is also formed.

Preferably, the present process is carried out at a temperature of from 20° C. to 150° C., in particular from 20° C. to 100° C., and at a pressure of from 1 bar absolute to 15 bars absolute, more preferentially from 1 bar to 7 bars, in particular from 2 to 3 bars.

Preferably, the compound A′ is introduced at stoichiometry or in excess with respect to the replacement number for bromine atoms which is desired in the compound A.

The organic solvent can be selected from the organic solvent S4 described above.

The process for the preparation of the compound B can also be carried out in the presence of H₂S. Preferably, the molar ratio H₂S/A′ is less than 0.4, more preferentially less than 0.3. In particular, the molar ratio H₂S/A′ is from 0.05 to 0.4, more particularly from 0.1 to 0.3. In a preferred embodiment, the compound A′ is NaSH and the molar ratio H₂S/NaSH is from 0.05 to 0.4, more particularly from 0.1 to 0.3.

The process for the preparation of the compound B can also be carried out with an oxygen content as described above in stage c) of the process according to the first aspect of the present invention.

The process for the preparation of the compound B can also comprise the stage c′) as described above for the process according to the first aspect of the present invention. The present process can thus make possible the preparation of a compound B of formula B-1, B-2, B-3, B-4, B-5, B-6, B-7, B-8 or B-9 as defined above in stage c) of the process according to the first aspect of the present invention. The compound B can also be purified as defined above in stage d) of the upgrading process according to the present invention.

### Compound B

According to a third aspect, the present invention relates to a compound B selected from the group consisting of the compounds B-1, B-2, B-3, B-4, B-5, B-6, B-7, B-8 and B-9 as defined above in which each R substituent is independently selected from —SH, —Br, —OH, —H, ═S or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;

preferably, in which each R substituent is independently selected from —SH, —Br, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;
more preferentially, in which each R substituent is independently selected from —SH, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH;
in particular, in which each R substituent is independently selected from —SH or —OH; provided that at least two R substituents are —SH.

According to a preferred embodiment, the compound B is the compound B-1 as defined above in stage c) of the process for the upgrading of brominated flame retardants.

According to another embodiment, the compound B is the compound B-2 as defined above in stage c) of the process for the upgrading of brominated flame retardants.

According to a preferred embodiment, the compound B is the compound B-3 in which at least two R substituents are —SH; preferably, the compound B is the compound B-3 in which at least three R substituents are —SH; more preferentially, the compound B is the compound B-3 in which at least four R substituents are —SH; in particular, the compound B is the compound B-3 in which at least five R substituents are —SH; more particularly, the compound B is the compound B-3 in which all the R substituents are —SH. When not all the R substituents are —SH, the remaining R substituents of the compound B-3 are independently selected from —OH and Br; preferably —OH.

According to another embodiment, the compound B is the compound B-4 in which at least two R substituents are —SH; preferably, the compound B is the compound B-4 in which at least three R substituents are —SH; more preferentially, the compound B is the compound B-4 in which all the R substituents are —SH. When not all the R substituents are —SH, the remaining R substituents of the compound B-4 are independently selected from —Br, —OH or form a C═C double bond, by a reaction for elimination of H—R with R═OH or Br, between the carbon atom carrying the R substituent and a carbon atom adjacent to the latter carrying a hydrogen atom; preferably are independently selected from —OH or form a C═C double bond, by a reaction for elimination of H—R with R═OH or Br, between the carbon atom carrying the R substituent and a carbon atom adjacent to the latter carrying a hydrogen atom; in particular are —OH.

According to a preferred embodiment, the compound B is the compound B-5 in which at least two R substituents are —SH; preferably, the compound B is the compound B-5 in which at least three R substituents are —SH; more preferentially, the compound B is the compound B-5 in which at least four R substituents are —SH; in particular, the compound B is the compound B-5 in which all the R substituents are —SH. When not all the R substituents are —SH, the remaining R substituents of the compound B-5 are independently selected from —OH and Br; preferably —OH.

According to a preferred embodiment, the compound B is the compound B-6 as defined above in stage c) of the process for the upgrading of brominated flame retardants.

According to a preferred embodiment, the compound B is the compound B-7 in which at least two R substituents are —SH; preferably, the compound B is the compound B-7 in which at least three R substituents are —SH; more preferentially, the compound B is the compound B-7 in which at least four R substituents are —SH; in particular, the compound B is the compound B-7 in which at least five R substituents are —SH; more particularly, the compound B is the compound B-7 in which at least six R substituents are —SH; favorably, the compound B is the compound B-7 in which at least seven R substituents are —SH; advantageously favorably, the compound B is the compound B-7 in which all the R substituents are —SH. When not all the R substituents are —SH, the remaining R substituents of the compound B-7 are independently selected from —OH and Br; preferably-OH.

According to a preferred embodiment, the compound B is the compound B-8 in which at least two R substituents are —SH; preferably, the compound B is the compound B-8 in which at least three R substituents are —SH; more preferentially, the compound B is the compound B-8 in which at least four R substituents are —SH; in particular, the compound B is the compound B-8 in which all the R substituents are —SH. When not all the R substituents are —SH, the remaining R substituents of the compound B-8 are independently selected from —OH and Br; preferably —OH.

According to a preferred embodiment, the compound B is the compound B-9 in which all the R substituents are —SH.

### Use of the Compound B

According to a fourth aspect, the present invention relates to the use of the compound B according to the present invention as crosslinking or vulcanizing agent, reactant for the preparation of sulfur-based compounds, such as thiourethanes, polysulfides and the like, chain-transfer agent, metal-complexing agent, ore flotation agent, antioxidant or heat stabilizer.

The present patent application also relates to the use of the compounds B obtained by the process according to the invention, as crosslinking agents in the preparation of adhesives, glues, mastics or coatings of the type of epoxy resins, acrylates, isocyanates and the like.

The compounds B obtained by the process according to the invention can also be used as reactants in thiol-ene reactions. This is because the presence of sulfhydryl groups having variable reactivity makes it possible to adjust the kinetics of the addition reaction of the thiol functional group to a diene group.

The compounds B obtained by the process according to the invention can also be used for the preparation of thiourethanes. This is because, by reacting polythiols, the content of thiol functional groups of which is controlled, thiourethane compounds are obtained with a chemical structure and performance qualities which are different from those which would have been obtained from polythiols synthesized either by the radical route or by the acid catalytic route which are conventional.

The compounds B obtained by the process according to the invention can also be used for the synthesis of polysulfides. This is because, by oxidation with sulfur, it is possible to obtain polysulfide compounds which can be used as additives for lubricants or for rubber. With the polythiols obtained by the process according to the invention, because of the difference in steric hindrance at the sulfur-based bonds, the polysulfides formed offer variable reactivities.

The compounds B obtained by the process according to the invention can act as chain-transfer agents during the synthesis of polymers from monomers, such as, for example, vinyl monomers, conjugated diene monomers, acrylic monomers, methacrylic monomers, and the mixtures of two or more of them in all proportions. The difference in reactivity depending on the type and the content of sulfhydryl group makes possible improved control of the polymerization reaction. As other use, the compounds B obtained by the process of the present invention can also be used as crosslinking agents for natural, artificial or synthetic rubbers, for unsaturated polymers (unsaturated polyesters), as metal-complexing agents, ore flotation agents, as oxygen scavengers, as corrosion inhibitors and the like.

1-18. (canceled)

19. A process for the upgrading of brominated flame retardants comprising the stages of:
a) provision of a plastic comprising a flame retardant A comprising a carbon-based chain substituted by at least two bromine atoms;
b) recovery of a composition comprising said flame retardant A;
c) bringing said composition comprising said flame retardant A into contact, in an organic solvent, with a compound A′ selected from the group consisting of Na₂S₂, K₂S₂, a potassium or sodium alkyl xanthate, and a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl, provided that R¹, R² and R³ are not simultaneously H; in order to form a mixture C comprising a compound B comprising a carbon-based chain substituted by at least two —SH functional groups; and
d) purification of the mixture C obtained and recovery of said compound B.

20. The process of claim 19, wherein the compound A′ is a compound of formula Y—SH in which Y is an alkali metal, an alkaline earth metal, a metal selected from rare earth metals, a —C(NH₂)═NH unit or a —C(NR¹R²)═NR³ unit with R¹, R² and R³ being, independently of one another, H or C₁-C₁₀ alkyl, provided that R¹, R² and R³ are not simultaneously H.

21. The process of claim 19, wherein the plastic comprises a thermosetting polymer selected from the group consisting of polyester, polyurethane, polyurea/polyurethane, phenol-formaldehyde resin, melamine resin, epoxy resin and polyimide; or the plastic comprises a thermoplastic polymer selected from the group consisting of poly(methyl methacrylate), acrylonitrile/butadiene/styrene (ABS), polyamide, polylactic acid, polyhydroxyalkanoate, polybenzimidazole, polycarbonate, polyethersulfone, polyoxymethylene, polyetheretherketone, polyetherimide, polyethylene, polyphenylene oxide, polyphenylene sulfide, polypropylene, polystyrene, poly(vinyl chloride), polyvinylidene fluoride (PVDF) and polytetrafluoroethylene (PTFE).

22. The process of claim 19, wherein stage b) comprises a stage of depolymerization or of dissolution of said plastic in order to recover a composition comprising said flame retardant A.

23. The process of claim 19, wherein stage c) is carried out at a temperature of from 20° C. to 150° C. and at a pressure of from 1 bar absolute to 15 bars absolute.

24. The process of claim 19, wherein the compound A′ is NaSH or KSH.

25. The process of claim 19, wherein stage c) is carried out in the presence of H₂S.

26. The process of claim 19, wherein stage c) is carried out in a reactor and, prior to bringing said flame retardant A into contact with said compound A′, a stream comprising an inert gas is introduced into the reactor.

27. The process of claim 19, wherein said carbon-based chain of said flame retardant A comprises from 3 to 20 carbon atoms.

28. The process of claim 19, wherein said flame retardant A is a C₃-C₂₀ alkyl at least substituted by two bromine atoms, C₃-C₂₀ alkenyl at least substituted by two bromine atoms, C₃-C₂₀ alkynyl at least substituted by two bromine atoms, C₃-C₂₀ cycloalkyl at least substituted by two bromine atoms, C₃-C₂₀ cycloalkenyl at least substituted by two bromine atoms, C₆-C₂₀ aryl at least substituted by two bromine atoms or C₃-C₂₀ ether at least substituted by two bromine atoms.

29. The process of claim 19, wherein said flame retardant A is selected from the group consisting of the compounds A-1, A-2, A-3, A-4, A-5, A-6, A-7, A-8 and A-9

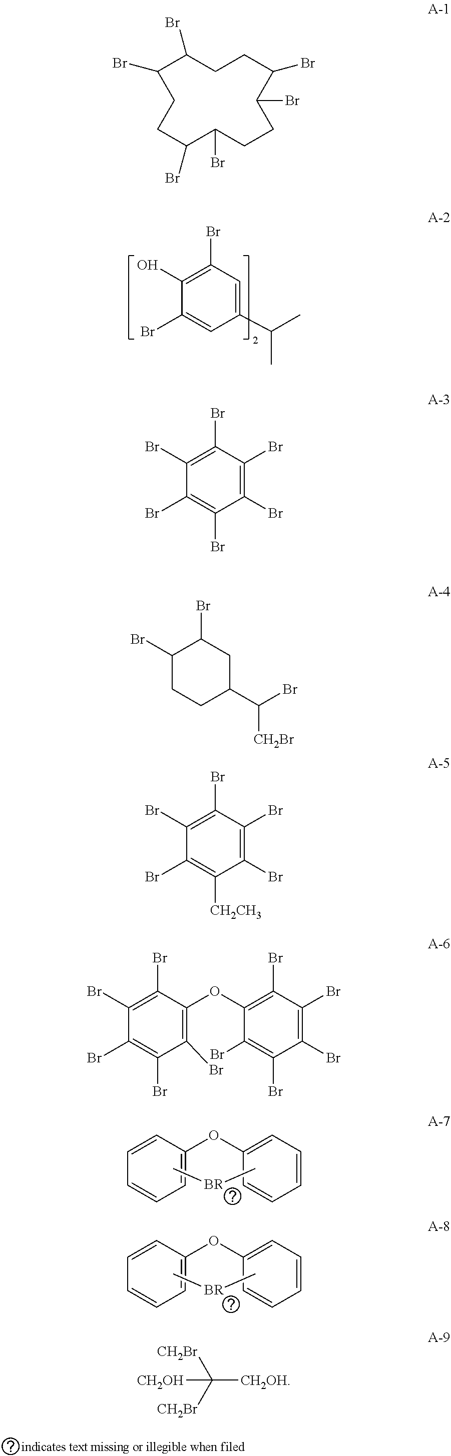

30. The process of claim 19, wherein said compound B is selected from the group consisting of the compounds B-1, B-2, B-3, B-4, B-5, B-6, B-7, B-8 and B-9 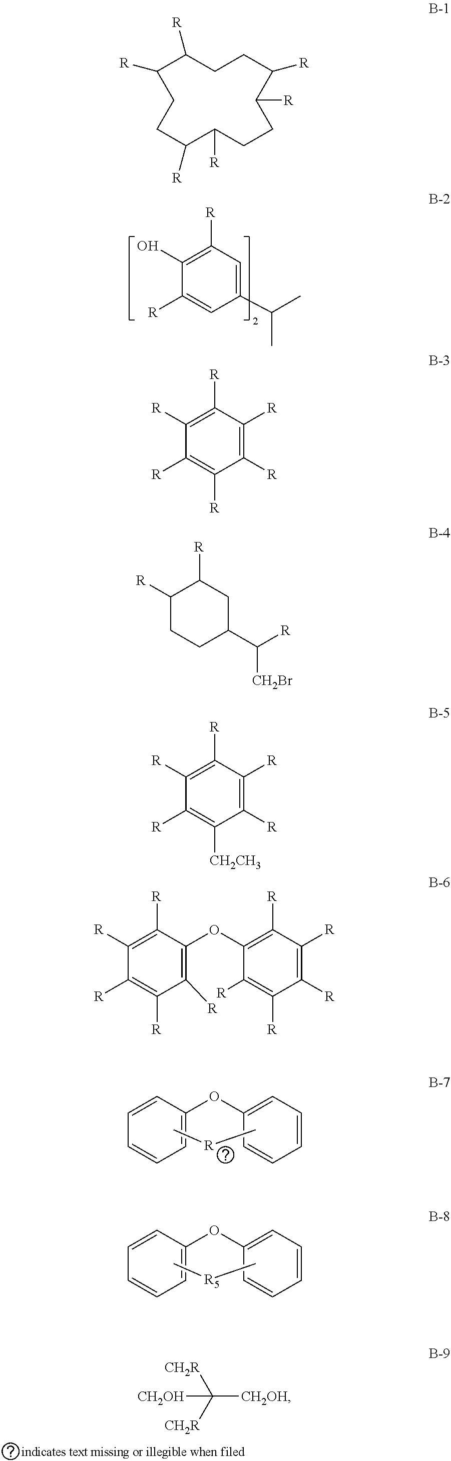
wherein at least two R substituents are —SH; the other R substituents being, independently of one another, —SH, —Br, —OH, —H, ═S or forming a C═C double bond, by a reaction for elimination of H—R with R═OH or Br, between the carbon atom carrying the R substituent and a carbon atom adjacent to the latter carrying a hydrogen atom; and
wherein, the other R substituents are, independently of one another, —SH, —Br, —OH or forming a C═C double bond, by a reaction for elimination of H—R with R═OH or Br, between the carbon atom carrying the R substituent and a carbon atom adjacent to the latter carrying a hydrogen atom.

31. The process of claim 19, wherein said flame retardant A is the compound A-1 and the compound B is the compound B-1 wherein at least two R substituents are —SH.

32. A process for the preparation of a compound B comprising at least two —SH functional groups, wherein compound B is selected from the group consisting of the compounds B-1, B-2, B-3, B-4, B-5, B-6, B-7, B-8 and B-9 as defined in claim 30 wherein at least two R substituents are —SH, the process comprises bringing a compound A comprising a carbon-based chain substituted by at least two bromine atoms into contact, in an organic solvent, with a compound A′ selected from the group consisting of Na₂S₂, K₂S₂, a potassium or sodium alkyl xanthate, and a compound of formula Y—SH wherein Y is an alkali metal, an alkaline earth metal, a metal selected from rare earth metals, a —C(NH₂)═NH unit or a —C(NR¹R²)=NR³ unit with R¹, R² and R³ being, independently of one another, H or C₁-C₁₀ alkyl, provided that R¹, R² and R³ are not simultaneously H;
wherein said compound A is selected from the group consisting of the compounds A-1, A-2, A-3, A-4, A-5, A-6, A-7, A-8, and A-9

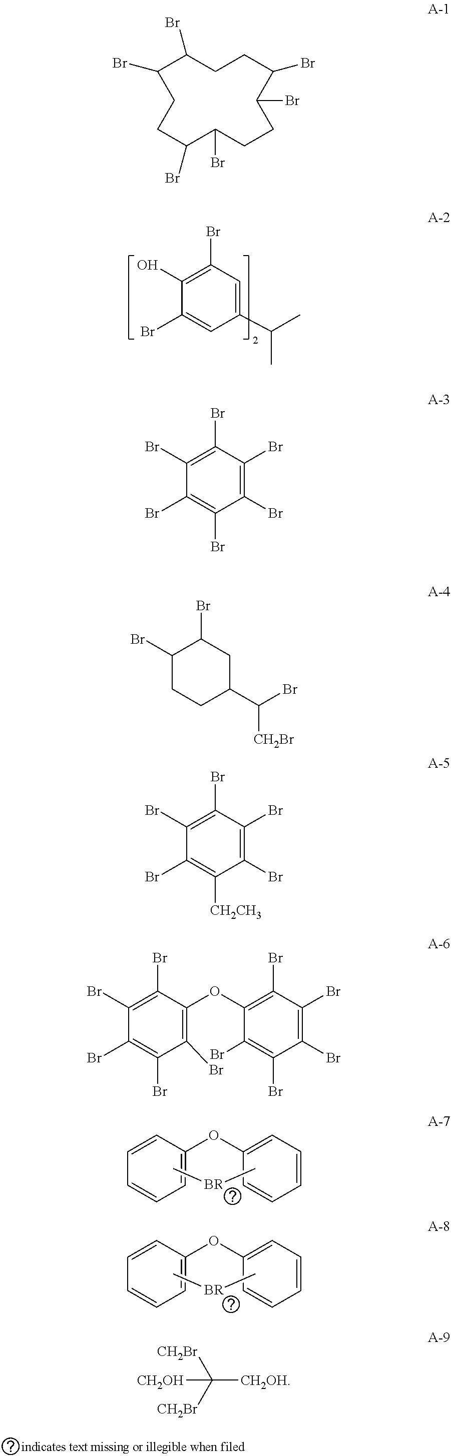

33. A compound B selected from the group consisting of the compounds B-1, B-2, B-3, B-4, B-5, B-6, B-7, B-8 and B-9 as defined in claim 30 in which each R substituent is independently selected from —SH, —Br, —OH, —H, ═S or forms a C═C double bond, by a reaction for elimination of H—R with R═OH or Br, between the carbon atom carrying the R substituent and a carbon atom adjacent to the latter carrying a hydrogen atom; provided that at least two R substituents are —SH.

34. The compound B of claim 33, wherein the compound is selected from the group consisting of the compounds B-1 and B-3 wherein each R substituent is independently selected from —SH, —Br, —OH or forms a C═C double bond, by a reaction for elimination of H—R with R═OH or Br, between the carbon atom carrying the R substituent and a carbon atom adjacent to the latter carrying a hydrogen atom; provided that at least two R substituents are —SH.

35. The compound B of claim 33, wherein the compound is selected from the group consisting of the compounds B-1, B-2, B-3, B-4, B-5, B-6, B-7 B-8 and B-9 wherein the R substituent is —SH.
